(19) United States
(12) Patent Application Publication
(10) Pub. No.: US 2009/0110706 A1
(43) Pub. Date: Apr. 30, 2009

(54) PAIN RELIEF COMPOSITION AND UTILIZATION THEREOF

(76) Inventors: Yoshihiro HIRATA, Kyoto city (JP); Yoshio UEDA, Kyoto city (JP); Kazuaki SUZUKI, Kyoto city (JP)

Correspondence Address: KNOBBE MARTENS OLSON & BEAR LLP, 2040 MAIN STREET, FOURTEENTH FLOOR, IRVINE, CA, 92614, US

(21) Appl. No.: 12/261,625

(22) Filed: Oct. 30, 2008

(30) Foreign Application Priority Data
Oct. 31, 2007 (JP) 2007-284221

(51) Int. Cl. A61K 33/24; A61P 25/00

(52) U.S. Cl. 424/402; 424/617

(57) ABSTRACT

A titanium-impregnated tape uses a material treated by fine dispersion water containing citric acid or its salt in which fine metal titanium particles are dispersed. The titanium-impregnated tape provides a physiological structure that acts upon the function to interfere with the pain memory system affecting neurons.

### BACKGROUND OF THE INVENTION

1. Field of the Invention

The present invention relates to a pain relief composition prepared from fine dispersion water of metal titanium, pain relief member obtained by treating a fiber material or resin material with such composition, and method for production thereof.

2. Description of the Related Art

Reflecting the recent trend of growing consumer awareness of health and sanitation, products designed to promote health or enhance cleanliness are drawing the attention. Particularly in the fields of food, clothing, articles of daily use, etc., the demand and need for these products are increasing. Among others, this need is strong especially for clothes (textile products) because of their sheer nature of being in constant contact with the body, where properties to promote health, prevent diseases, or enhance cleanliness, are considered effective.

In light of this situation, a number of products have been developed through active studies on healthy clothes. For example, clothes that use fiber materials, carbon materials or other materials generating far infrared light or magnetic force to treat diseases are already commercialized, while other products are also drawing the attention such as fiber products designed to relieve fatigue by using the effect of tourmaline, as well as clean, antibacterial fiber products utilizing inorganic compounds, chitosan, etc.

Metal titanium is a metal material discovered relatively recently compared to iron, copper, aluminum, etc. Being lightweight and exhibiting sufficient strength at high temperatures, metal titanium is used in various industrial fields such as the aerospace industry where titanium is used in jet engines, etc. In energy fields, metal titanium is a material for heat exchanger tubes and tube plates used at nuclear power stations and thermal power stations, among others. Metal titanium is also used in a wide range of articles of daily use including eyeglass frames and golf club heads. The application fields of metal titanium are expanding further.

Applications of metal titanium in articles of daily use, healthcare/medical products and cosmetics are relatively large in number. Examples include hair scissors whose surface is coated with a thin titanium film (Japanese Patent Laid-open No. Sho 62-268584), molten metal titanium materials that facilitate utilization of far infrared light (Japanese Patent Laid-open No. Hei 1-155803, Japanese Patent Laid-open No. Hei 3-112849), linen (Japanese Patent Laid-open No. Hei 8-322695), cooking equipment (Japanese Patent Laid-open No. Hei 9-140593), eye mask (Japanese Patent Laid-open No. Hei 10-71168), health-maintaining equipment (Japanese Patent Laid-open No. Hei 11-285541, Japanese Patent Laid-open No. Hei 11-285543), health band (Japanese Utility Model Registration No. 3045835), and health slippers (Japanese Utility Model Registration No. 3061466), among others.

As for water-based dispersion liquid in which fine metal titanium particles are dispersed, a method is known to produce high-function water in which titanium is dissolved, which is characterized in that a mixture gas of oxygen and hydrogen is burned in high-pressure water and the resulting combustion gas is used to melt metal titanium (Patent Literature 1).

As a product applying the above technology, a clothing material is also known that is obtained by soaking a fiber material in an aqueous solution containing molten titanium obtained by burning metal titanium using the combustion gas generated from burning oxygen and hydrogen in high-pressure water (Patent Literature 2), as well as a skin lotion made from titanium dispersion liquid produced by the same method (Patent Literature 3).

On the other hand, dispersion liquids containing titanium oxide, not dispersion liquids containing element titanium, are also known. Examples include a yarn, cloth or non-woven fabric to which the photo-catalytic activity of titanium is added by impregnating it with a mixture solution containing a dye and an aqueous titanium oxide solution (Patent Literature 4), and a base fiber material to which such functions as UV absorbency, deodorizing property and antibacterial property are added by impregnating it with a fine aqueous dispersion liquid containing sericin-titanium oxide (Patent Literature 5).

Furthermore, regarding the utilization of titanium components in medical fields some applications are known in which titanium is used in treatment implements that are attached to the body surface. Examples include use of a fiber product, etc., impregnated with an aqueous titanium oxide solution as a treatment cloth to treat various diseases, etc., specifically working as an anti-inflammatory agent, hemostatic agent, treatment drug for burns, or drug to improve cuts, abrasions, dermatitis, etc. (Patent Literature 6), and use of a stick made by mixing and kneading fine titanium material particles and adhesive to make a thin piece that can be attached to the body to heal bruise, sprain, stiff shoulder, muscle pain, etc. (Patent Literature 7). However, the mechanism of operation of each of these products is unclear.

The human body consists of numerous cells that are divided in blocks to govern breathing, circulation, digestion, metabolism and various other functions of the body. The nerve system controls the tissues of these organs, and this nerve system is structurally divided into the brain, spinal cord and peripheral nerves. Among these, the brain and spinal cord make up the central nerves (central nerve system) functioning as a control system that receives information sent from peripheral nerves and sends instructions according to the received information. Peripheral nerves provide a communication network that extends throughout the body, and this network sends information to the central nerves and also transmits instructions from the central nerves. Functionally, the nerve system is divided into somatic nerves (somatic nerve system) and autonomic nerves (autonomic nerve system), where somatic nerves function as receivers that transmit to the brain the image, sound, sensation of touch and other information regarding actions to have the information recognize by the brain, and also as transmitters that convey instructions to move the various parts of the body in response to this information. Those nerves that act as receivers are called the perception nerve system, while others that act as transmitters are called the motor nerve system.

Incidentally, so-called pain relief members and pain relief compositions used in pain relief members, designed to adjust the effects on perception nerves or relieve the pain felt by perception nerves when applied near the painful area, were not heretofore known.

[Patent Literature 1] Japanese Patent Laid-open No. 2001-314871

[Patent Literature 2] Japanese Patent Laid-open No. 2002-20969

[Patent Literature 3] Japanese Patent No. 3715301

[Patent Literature 4] Japanese Patent Laid-open No. 2002-180385

[Patent Literature 5] Japanese Patent Laid-open No. 2006-342477

[Patent Literature 6] Japanese Patent Laid-open No. 2001-106633

[Patent Literature 7] Japanese Patent No. 2935974

### SUMMARY OF THE INVENTION

As explained above, there are hopes for considerable growth in the applications of titanium in bioactive materials, medical products, etc., and some products are already developed. However, the operations and effects titanium has on physiological mechanisms are mostly unknown.

As one way to realize the almost infinite possibilities of titanium, the present invention aims to utilize a composition constituted by fine dispersion water containing titanium in the medical field. In particular, it is the object of the present invention to provide a composition that adjusts the effects on the nerve system or specifically demonstrates an action to relieve pain, as well as a product that uses such composition.

To be specific, the basic constitution of the present invention is explained as follows:

1) A pain relief composition whose major component is fine dispersion water containing organic acid (salt), constituted by fine metal titanium particles dispersed in water.

2) A pain relief composition according to (1), characterized in that the organic acid (salt) contains at least one component selected from citric acid (salt), L-ascorbic acid (salt) and L-sorbic acid (salt).

3) A pain relief composition according to (1) or (2), characterized in that the concentration of the organic acid (salt) is in a range of 0.01 to 1%.

4) A pain relief member characterized in that its material is treated by fine dispersion water according to any one of (1) to (3) above.

5) A pain relief member according to (4), characterized in that its material is non-woven fabric or woven fabric or porous resin.

6) A pain relief product characterized in that the product is selected from panty hoses, socks, gloves, underwear, shirts, linen, healthy athletic wear, mufflers, towels, supporters or wrist bands, construction materials, wall materials, necklaces, tapes (including taping tapes), adhesive bandages, elastic bandages, gauzes, eye-patches, sanitary items, poultices, clothes, etc., that use a member according to (5).

### BRIEF DESCRIPTION OF THE DRAWINGS

FIG. 1 is a figure plotting the degree of slope of excitatory post-synaptic potential relative to elapsed time in order to observe long-term potentiation (LTP).

FIG. 2 is a figure plotting the voltage of resting potential membrane for each sample.

FIG. 3 is a figure plotting the frequency at which ignition occurs in each sample due to action potential.

FIG. 4 is a flow of production of titanium fine dispersion water conforming to the present invention.

FIG. 5 is an apparatus for producing titanium fine dispersion water conforming to the present invention.

### DETAILED DESCRIPTION OF THE PREFERRED EMBODIMENTS

Under the present invention, the base material impregnated with fine dispersion water, where such water contains citric acid (salt) and is constituted by fine metal titanium particles dispersed in water, has a physiological mechanism that acts upon the function to interfere with the pain memory system of nerve cells, especially neurons that are particularly sensitive, and specifically provides a superior action to relieve pain. Also, the base material treated by titanium fine dispersion water as proposed by the present invention has an excellent effect in that it has no toxicity on neurons.

The fine dispersion water constituted by fine metal titanium particles as proposed by the present invention further advances the method for producing fine dispersion water constituted by fine metal titanium particles that was filed earlier by the applicant, and further improves the stability of the highly stable titanium dispersion liquid obtained by the aforementioned earlier method.

As described in the Specification included in the application for patent filed by the applicant, fine dispersion water in which fine metal titanium particles are dispersed can be easily obtained by burning metal titanium using the high temperature generated by the combustion gas, which in turn is produced by burning oxygen and hydrogen in high-pressure water inside a pressure container (Japanese Patent No. 3715301, Japanese Patent No. 3686819; U.S. Pat. No. 6,989,127, No. 7,108,735, No. 7,118,684, No. 7,144,589, No. 7,300,491, No. 7,300,672, and No. 7,320,713, U.S. Patent Publication No. 2005/0008585, the disclosure of each of which is herein incorporated by reference in its entirety for producing titanium fine particles and its dispersion water).

The base material treated by high-function titanium fine dispersion water obtained by the present invention has a physiological mechanism that acts upon the function to interfere with the pain memory system of nerve cells, especially neurons that are particularly sensitive, and specifically provides a superior action to relieve pain, while having no toxicity on neurons.

Regarding the base material treated by high-function titanium fine dispersion water obtained by the present invention, the mechanism of why it provides the aforementioned excellent bioactive efficacies is unclear. However, the inventor is currently studying diligently to uncover the scientific grounds for these functions.

The titanium fine dispersion water proposed by the present invention can be produced, for example, by the method disclosed in Japanese Patent No. 3715301. According to this production method, metal titanium is melted, not by using a common, general-purpose means for melting metal, but by using the combustion heat from oxygen and hydrogen, and then molten titanium is dispersed into high-pressure water as superfine titanium particles. Production of such dispersion water is implemented using a production apparatus explained below.

Basically, hydrogen and oxygen are burned and then a pure metal titanium bar is inserted into the atmosphere of combustion gas to heat the bar and cause molten titanium to collide with high-pressure water, thereby dispersing superfine titanium particles in water.

Under this production method, excellent bioactivity is added to the product. Accordingly, the amounts of hydrogen and oxygen to be burned, reaction pressure, and amount of metal titanium to be supplied, must be controlled.

The method for producing titanium fine dispersion water in high-pressure water, proposed by the present invention, is explained using drawings.

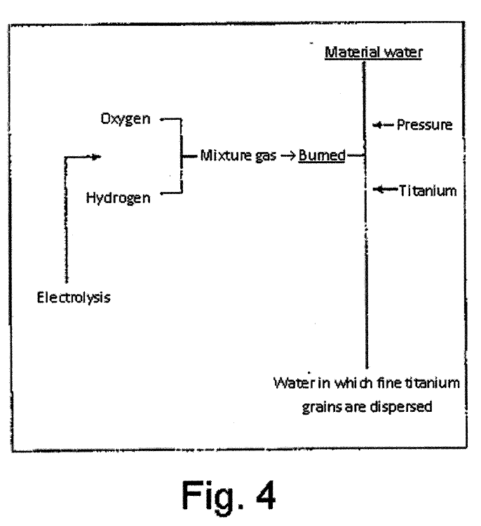

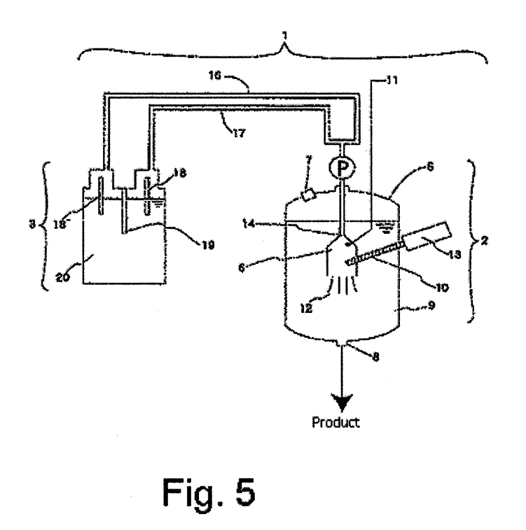

FIG. 4 is a flowchart of how titanium fine dispersion water is produced according to the present invention, while FIG. 5 shows an apparatus for producing a skin lotion constituted by titanium fine dispersion water according to the present invention.

A production apparatus for titanium fine dispersion water 1 shown in FIG. 5 comprises a pressure container 2 for producing high-pressure water in which molten titanium is dispersed, an electrolysis/material-gas generating device 3, and a filtering device (not illustrated) for filtering titanium fine dispersion water.

The pressure container 2 is a device for producing titanium fine dispersion water in which superfine titanium particles are dispersed, where the device comprises a high-pressure water tank 5, an oxygen/hydrogen mixture-gas injection nozzle 14, a combustion chamber 6, and a metal titanium bar 10. As attachments, the apparatus has the water electrolysis device 3 to supply material hydrogen and oxygen, and the filtering system to filter the produced titanium fine dispersion water.

Also, the pressure container 2 has the high-pressure water tank 5 made of metal, or preferably steel. In the high-pressure water tank 5, mixture gas of hydrogen and oxygen generated by the electrolysis device 3 and supplied through a hydrogen supply channel 16 and an oxygen supply channel 17 is injected under high pressure into the combustion chamber 6 from the injection nozzle 14. Inside the combustion chamber 6, the metal titanium bar 10 is gradually introduced into the combustion chamber 6 from a supply cylinder 13 according to the melting amount. The mixture gas of hydrogen and oxygen is ignited by an ignition device 11, and molten metal titanium is released into high-pressure water 9. Molten titanium becomes superfine particles in high-pressure water, and then high-pressure water 9 containing these particles is taken out of the apparatus from an outlet 8 at the bottom of the high-pressure water tank to be filtered sequentially through an appropriate filtering device.

Here, it is possible to use high-pressure cylinders each containing hydrogen and oxygen as the material-gas generating device 3 to directly supply hydrogen and oxygen into the high-pressure water tank 5. However, oxygen and hydrogen supplied under the present invention are produced by electrolysis of water and therefore 100% pure. Accordingly, fuel gas having a theoretical oxygen/hydrogen mixture ratio of 1:2 can be supplied efficiently to shut out any components other than titanium from the fine dispersion matrix.

As for the material-gas generating device 3 for generating the material for producing titanium fine dispersion water, the illustrated device provides one example where hydrogen and oxygen are generated by electrolysis of water 20, where numerals 18 and 18′ indicate a cathode plate and an anode plate, respectively.

In this apparatus, hydrogen and oxygen generated by electrolysis and supplied through the hydrogen supply channel 16 and oxygen supply channel 17 are injected into the combustion chamber 6 from the nozzle 14 by means of a pump, and the resulting mixture gas is fully burned to produce perfect ultrahigh-temperature gas of combustion steam. Then, the pure metal titanium bar 10 is inserted into this combustion gas to heat and melt the bar. The metal titanium bar is inserted from the cylinder 13 by a fixed amount at a time according to the melting amount. When melting the metal titanium bar, the mixture ratio of hydrogen and oxygen must be strictly controlled to 2:1. Also, a pressure regulation valve 7 must be provided to adjust the pressure in the high-pressure water tank.

Molten titanium 12 that has been heated to high temperature and thus melted in the combustion chamber 6 is released into high-pressure water 9 from the combustion chamber 6, where it collides with high-pressure water to generate superfine particles. At this time, a part of titanium is considered to form a crystalline structure.

Because superfine titanium particles generate in water in this condition, these superfine titanium particles have very high hydrophobicity and become dispersed in water in a stable manner. Accordingly, these particles do not settle, even when a coagulating agent is used.

This apparatus is operated as follows. First, high-pressure hydrogen/oxygen is injected into the high-pressure water tank 5 from the nozzle 14 by means of a pump, and then ignited by the ignition device 11 to produce ultrahigh-temperature combustion steam gas, after which the pure metal titanium bar 10 is inserted gradually into this combustion gas to melt the bar.

With this apparatus it is essential to burn hydrogen and oxygen in water in order to prevent production of substances other than water and superfine titanium particles, and for this reason it is necessary to burn hydrogen and oxygen under high pressure so that they will burn fully in water without containing impurities. Also, the position at which the metal titanium bar is inserted must be in an area where the mixture gas burns completely to produce perfect ultrahigh-temperature steam gas.

When obtaining the titanium fine dispersion water proposed by the present invention using the aforementioned apparatus, significant effects can be achieved by adding organic acid (salt) as an electrolyte.

For example, in an experiment where titanium fine dispersion water was produced by adding citric acid and then the obtained water was filtered sequentially through filters of 2μ, 1μ and 0.8μ, the titanium concentration measured by quantitative analysis based on ICP emission spectrometry was approx. 70 mg/l. On the other hand, the titanium concentration of titanium fine dispersion water produced under the same conditions, but without adding citric acid, was approx. 0.4 mg/l.

This suggests that the titanium fine dispersion water produced without adding citric acid caused fine titanium particles to coagulate and thus become trapped by the filters, resulting in the lower titanium concentration.

This reveals that producing titanium fine dispersion water by adding citric acid, etc., leads to mitigation of coagulation and settling of particles, and consequently improved stability.

As for the organic acid (salt) added during production, citric acid (salt), L-ascorbic acid (salt) and L-sorbic acid (salt) can be considered, among others. Adding these acids increases the titanium content in metal titanium fine dispersion water and improves the stability of titanium in dispersion water. In particular, preferably this organic acid should be citric acid or salt thereof, having a pH value of 2 to 4, or preferably 2.2 to 2.7, as it is added during the production of the titanium fine dispersion water. The concentration of citric acid to be added should be 0.01 to 0.2 percent by weight, or preferably 0.1 to 1 percent by weight. It was also found that titanium in fine dispersion water would stabilize when polyvinyl pyrrolidone is added as a water-soluble polymer.

Under the present invention, titanium fine dispersion water produced by the production method mentioned above is used as a pain relief composition. As for the mode of use of this composition, impregnating a base material with titanium dispersion water causes metal titanium to coagulate and settle due to trace amounts of mineral components such as Na, Ca and Mg. Accordingly, the impregnation process requires expert knowledge and thus implementing it was possible only at special plants equipped with pure-water facility to produce purified water, etc., required in the process. However, it was found that, by using titanium fine dispersion water produced by adding citric acid (salt), etc., to water in the production process, the content of dispersed titanium in water would improve and titanium in water would also stabilize. As a result, it is now possible to produce a pain relief composition at any general plant having no special facility, etc., as mentioned above.

The base material to be treated, or impregnated for example, using the aforementioned fine dispersion water in which fine metal titanium particles are dispersed, must be a material that can be easily impregnated with and retain metal titanium. Accordingly, synthetic or natural non-woven fabric or woven fabric or porous resin material would be appropriate.

As for the forms of products using the aforementioned base material treated by fine dispersion water in which fine metal titanium particles are dispersed in water, examples include products selected from panty hoses, socks, gloves, underwear, shirts, linen, healthy athletic wear, construction materials, wall materials, necklaces, tapes (including taping tapes), adhesive bandages, elastic bandages, gauzes, eye-patches, sanitary items, poultices, clothes, mufflers, towels, supporters and wrist bands.

### EXAMPLE

The production apparatus disclosed in Japanese Patent No. 3715301 mentioned above (which corresponds to U.S. Patent Publication No. 2005/0008585) was used to prepare titanium fine dispersion water constituted by 99.84% purified water, 0.1% citric acid and 0.06% titanium (titanium concentration: 600 mg/l). This dispersion water was diluted with water as deemed necessary to prepare three water dispersion liquids of 7 mg/l, 100 mg/l and 600 mg/l in concentration, as specified below.

The obtained water dispersion liquids were each impregnated in a plain-weave base material (33% cotton, 61% polyester, 6% polyurethane) to produce the samples shown in [Table 1].

Four samples were prepared, having incremental titanium concentrations with Sample No. 1 having the lowest concentration.

TABLE 1

| Sample No. | Base material | Titanium concentration* | |
|---|---|---|---|
| 1 | Plain-weave fabric (dyed black) | 0 | |
| 2 | Plain-weave fabric (dyed black) | 7 | mg/l |
| 3 | Plain-weave fabric (dyed black) | 100 | mg/l |
| 4 | Plain-weave fabric (dyed black) | 600 | mg/l |

*The titanium concentration was measured as the mg content of element titanium per 1 liter of water.

<Purpose of Experiment>

The above samples were measured as follows to check if the present invention would produce a physiological structure to relieve pain.

<Method of Experiment>

It is known in the field of cerebral neurology that the physiological reaction to pain can be checked by measuring the electrical changes occurring in hippocampal cells, which control pain-sensing nerves in the brain. Also, it is difficult to prepare human samples for this type of experiment, and therefore hippocampal cells from laboratory animals, or specifically mice, were used. In the field of cerebral neurology, results obtained from mouse cells have been shown to structurally exhibit certain repeatability in human.

Accordingly, hippocampal cells from mice were sliced and soaked in temperature-controlled artificial cerebrospinal fluid in order to recreate a bioactive environment. To be specific, hippocampal cells were connected to electrodes to recreate an environment where pain-sensing nerves would be stimulated. Each of the following samples was placed not in contact with, but in the immediate vicinity of, hippocampal cells to perform the following measurements. The obtained samples conforming to the present invention, prepared by the aforementioned production method, were identified as Samples 1 to 4 as shown in [Table 1] according to their titanium concentrations.

To be specific, in cerebral neurology the following three items are recognized as objective indicators having repeatability that can be used in the measurement of reaction of hippocampal cells to pain. Accordingly, these items were measured on samples having varying titanium concentrations from none, or 0 mg/l, to higher concentrations up to 600 mg/l as obtained by the aforementioned production method, in order to see if there were significant differences. These items can be measured by recording the condition of electricity generated when hippocampal cells are stimulated electrically, and therefore Samples 1 to 4 of the present invention as mentioned above were placed in the immediate vicinity of hippocampal cells and the hippocampal cells were stimulated electrically to record the condition of generated electricity.

LTP (Synaptic Plasticity):

LTP (long-term potentiation) refers to a phenomenon where application of continuous stimulations at high frequency to the neurites of pre-synaptic neurons achieves a greater excitatory post-synaptic potential (EPSP) to increase the efficiency of synaptic transmission over a long period. Here, it can be said that the faster the ratio to the normal LTP drops, the faster the nerve cells are restored to a normal condition.

Resting Membrane Potential:

Ions continue to move and flow into/out of cells as long as the cells are alive. However, movement of electric charges apparently stops under a certain condition, and the membrane potential that provides this condition is called the resting membrane potential. It is known that the lower the resting membrane potential, the more stable nerve cells become in their resting state.

Action Potential Frequency:

When alternating current is applied to the synapses of hippocampal cells, they ignite at a certain frequency. This frequency is called the action potential frequency. It is said that the lower this frequency, the quicker the nerves are reacting to signals, indicating higher transmission efficiency.

<Results of Experiment>

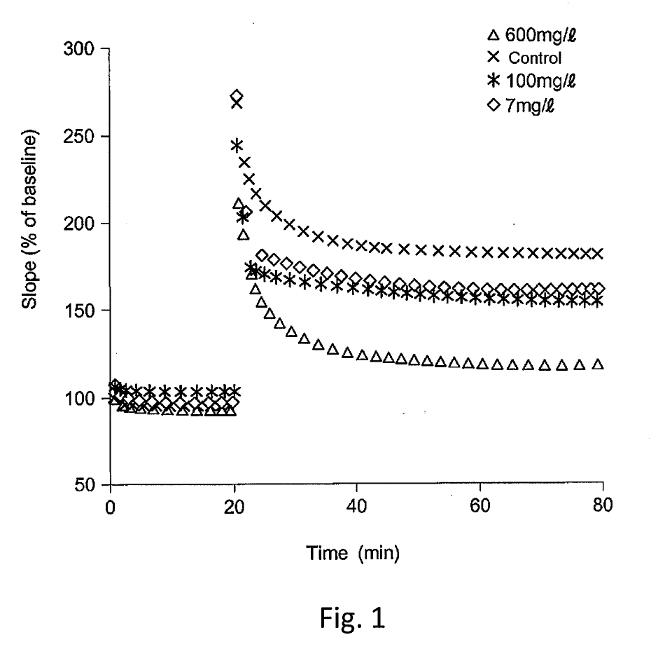

The results of this experiment are shown in FIGS. 1 to 3 based on measured results of (a) to (c).

Here, the electrical reactions of ATT samples (tapes impregnated with titanium fine dispersion water) at different concentrations are shown in graphs.

FIG. 1 shows the effect on LTP (a), where compared to the 0-mg/l sample (control) the samples impregnated with titanium fine dispersion water of 7 mg/l and 100 mg/l have relatively lower slope ratios in general, with the LTP dropping faster with an elapse of time in the latter samples. This trend is more prominent with the 600-mg/l sample.

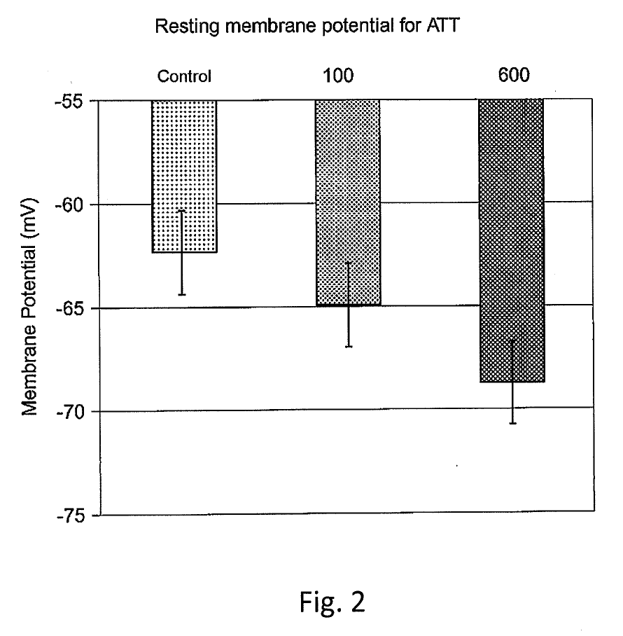

As for the resting membrane potential (b), as shown in FIG. 2 the 100-mg/l sample exhibits the resting membrane potential at a relatively lower voltage than the 0-mg/l sample (control). This trend is more prominent with the 600-mg/l sample.

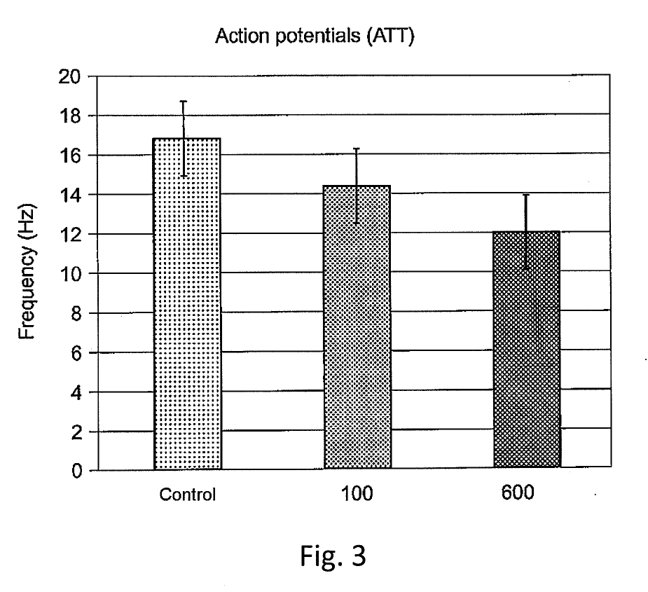

Furthermore regarding the action potential frequency (c), as shown in FIG. 3 the 100-mg/l sample has a relatively lower action potential frequency compared to the 0-mg/l sample (control). This trend is more prominent with the 600-mg/l sample.

[Overall Summary of Experiment]

The above results of experiment provide the following findings regarding the samples of respective titanium concentrations.

The titanium-impregnated tapes of 7 mg/l and 100 mg/l in titanium concentration have superior recoverability (=LTP dropping and stabilizing more quickly), greater stability in a resting state (=lower resting membrane potential), and improved signal reception sensitivity and transmission efficiency (=lower action potential frequency), compared to the 0-mg/l sample containing no titanium at all.

These trends are more prominent with the sample having a higher concentration of 600 mg/l.

The above results show that a pain relief composition conforming to the present invention would have the effects of enhancing the stability and transmission efficiency of nerve cells and consequently relieving pain, as observed in areas near the titanium-impregnated tapes respectively impregnated with titanium at 7 mg/l, 100 mg/l and 600 mg in concentration.

Furthermore, it has become clear that the titanium-impregnated tapes using the samples conforming to the present invention have a physiological structure that acts upon the function to interfere with the pain memory system affecting neurons, which are most sensitive cells in the human body, without having any toxicity on neurons.

In the present disclosure, “the invention” means one or more embodiments of the present invention. The term “an organic acid or its salt” means at least an organic acid or its salt and does not exclude a mixture of the organic acid and its salt.

This application claims priority to Japanese Patent Application No. 2007-284221, filed Oct. 31, 2007, the disclosure of which is herein incorporated by reference in its entirety.

1. A pain relief aqueous composition comprising, as a main component, titanium dispersion water containing an effective amount of fine metal titanium particles dispersed therein and an organic acid or its salt.

2. The pain relief aqueous composition according to claim 1, wherein the organic acid or its salt contains at least one component selected from citric acid or its salt, L-ascorbic acid or its salt, and L-sorbic acid or its salt.

3. The pain relief aqueous composition according to claim 1, wherein the concentration of the organic acid or its salt is in a range of 0.01 to 1%.

4. A pain relief member comprising a base material treated with the pain relief aqueous composition of claim 1.

5. The pain relief member according to claim 4, wherein the base material is non-woven fabric or woven fabric or porous resin.

6. A pain relief product comprising a product constituted by the pain relief member of claim 5, said product being selected from panty hoses, socks, gloves, underwear, shirts, linen, healthy athletic wear, mufflers, towels, supporters or wrist bands, construction materials, wall materials, necklaces, tapes including taping tapes, adhesive bandages, elastic bandages, gauzes, eye-patches, sanitary items, poultices, or clothes.

7. A method for relieving pain comprising:
providing a material containing or treated with titanium dispersion water containing an effective amount for relieving pain of fine metal titanium particles dispersed therein and an organic acid or its salt; and
placing the material in the vicinity of an area of pain.

8. The pain relief aqueous composition according to claim 2, wherein the concentration of the organic acid or its salt is in a range of 0.01 to 1%.

9. The pain relief member according to claim 4, wherein the organic acid or its salt contains at least one component selected from citric acid or its salt, L-ascorbic acid or its salt, and L-sorbic acid or its salt.

10. The pain relief member according to claim 9, wherein the concentration of the organic acid or its salt is in a range of 0.01 to 1%.

11. The pain relief product according to claim 6, wherein the organic acid or its salt contains at least one component selected from citric acid or its salt, L-ascorbic acid or its salt, and L-sorbic acid or its salt.

12. The pain relief product according to claim 11, wherein the concentration of the organic acid or its salt is in a range of 0.01 to 1%.

13. The method according to claim 7, wherein the organic acid or its salt contains at least one component selected from citric acid or its salt, L-ascorbic acid or its salt, and L-sorbic acid or its salt.

14. The method according to claim 13, wherein the concentration of the organic acid or its salt is in a range of 0.01 to 1%.
